SEQUENCE LISTING (8 sequences)

SEQ ID NO: 1 - DNA, 18 bp, Artificial Sequence
  Synthetic construct
gcaatgcactggagttcg

SEQ ID NO: 2 - DNA, 20 bp, Artificial Sequence
  Synthetic construct
acgatggacgtaagggagtg

SEQ ID NO: 3 - DNA, 20 bp, Artificial Sequence
  Synthetic construct
gcctgagcaaagtgaatgag

SEQ ID NO: 4 - DNA, 19 bp, Artificial Sequence
  Synthetic construct
gcagaccttcgatgtagcg

SEQ ID NO: 5 - DNA, 26 bp, Artificial Sequence
  Synthetic construct
catggagtcctggcatccacgaaact

SEQ ID NO: 6 - DNA, 26 bp, Artificial Sequence
  Synthetic construct
atctccttctgcatcctgtcggcata

SEQ ID NO: 7 - DNA, 20 bp, Artificial Sequence
  Synthetic construct
atgtttgtgatgggtgtgaa

SEQ ID NO: 8 - DNA, 20 bp, Artificial Sequence
  Synthetic construct
atgccaaagttgtcatggat

(19) United States
(12) Patent Application Publication
(10) Pub. No.: US 2023/0272345 A1
(43) Pub. Date: Aug. 31, 2023

(54) SELF-ASSEMBLED CELL SHEET CONSTRUCTS AND METHODS OF MAKING THEREOF

(71) Applicant: Caro Meats Inc., Kitchener (CA)

(72) Inventors: Ponnambalam Ravi Selvaganapathy, Dundas (CA); Alireza Shahin-Shamsabadi, Hamilton (CA)

(21) Appl. No.: 18/012,743

(22) PCT Filed: Jun. 30, 2021
(86) PCT No.: PCT/CA2021/050898

§ 371 Date: Dec. 23, 2022

Related U.S. Application Data
(60) Provisional application No. 63/046,370, filed on Jun. 30, 2020.

(51) Int. Cl. C12N 5/077; A23L 13/00

(52) U.S. Cl. CPC C12N 5/0658; C12N 5/0653; A23L 13/00; C12N 2509/00; A61F 2/022

(57) ABSTRACT

This application relates to a method of making a cell construct, comprising a) plating a plurality of cells on a substantially flat surface; b) growing the plurality of cells to at least 80% confluent to form a cell sheet with intercellular linkages; c) applying a culture medium having a pH of about 5 to about 6.8 to the cell sheet; d) replacing the culture medium of step c) with a culture medium having a pH of about 7.5 to about 8.5; and e) replacing the culture medium of step d) with a culture medium having a pH of about 7 to about 7.7, to obtain a substantially planar untethered cell sheet. Also provided is a cell construct formed according to the method and uses thereof.

### CROSS-REFERENCE TO RELATED APPLICATION

The present application claims the benefit of priority from co-pending U.S. provisional patent application No. 63/046,370 filed on Jun. 30, 2020, the contents of which is incorporated herein by reference in its entirety.

### FIELD

The present application relates to tissue engineering, and in particular, to the biofabrication of self-assembled cell sheet constructs.

### BACKGROUND

Regenerative medicine holds the promise to repair damaged and diseased organs or tissues through the implantation of externally cultivated cells, tissues or tissue like constructs. Direct injection of bioinks consisting of stem cells has been widely investigated due to their ease of use but have not shown efficacy. An alternative is to incorporate the cells in a synthetic or biodegradable scaffold which can be used to form a tissue-like construct to prevent loss of the cells and facilitate better integration with existing tissue. A more recent approach has been to form sheets of cells using self-assembly approaches that dispense away the need for synthetic scaffolds by inducing cells to secrete their own extracellular matrix (ECM) and preserving it to form tissue constructs that are ideally suited for integration with host tissue or organ (Matsuura, K., et al., 2014). This cell sheet engineering approach allows non-invasive and non-enzymatic harvest of cells, cell-cell and cell-extracellular matrix (ECM) junctions and the ECM that they have deposited as a continuous layer (Yamada, N., et al., 1990). If transplanted in vivo, cells within these sheets will be regulated according to host environment to secrete appropriate growth factors and cytokines. It could also be used as means for protein delivery to the body (Matsuura, K., et al., 2014).

Cell sheet engineering was first introduced in 1990s using cell culture dishes that were grafted with N-isopropylacrylamide (PIPAAm) which enabled detachment of cells and their secreted proteins without enzymatic treatment in an intact format just by decreasing the temperature and changing the hydrophilicity of the grafted polymer (Yamada, N., et al., 1990). The cells in these sheets show higher viability as the secreted proteins, such as E-cadherin and Laminin 5 (Yamato, M., et al., 2001) are preserved and the continuous sheets have adhesive properties such that they can be stacked on top of each other or other tissues and adhere to without using glue or suture (Iwata, T. et al., 2011). Alternatively, special surfaces created by layer-by-layer deposition of cationic and anionic polyelectrolytes on indium tin oxide have also been used to trigger delamination of cell sheets as these layers become unstable at low pH (Guillaume-Gentil, O., et al., 2011). In this technique pH of the medium needs to be around 4 for cell detachment process to start (detachment starts after 2-3 min) which may affect cell viability. Other techniques that allow formation of continuous cell sheets include growing cells on a feeder layer and detaching the cell sheet using dispase that digests some of the ECM proteins and not cell-cell junctions, growing cells on amniotic membrane as a carrier and then using them with this membrane, or initiating the delamination through application of external stimuli such as light, electrochemical polarization, ionic solution, and magnetic force (Owaki, T., et al., 2014). It has been shown that the delamination process for some of these techniques could be very long, for example in case of PIPAAm-grafted surfaces a 40-80 min incubation time at 20° C. might be necessary (Tang, Z. et al., 2012) which could have adverse effect on cell viability. All of these methods involve specialized surfaces for culture or use of enzyme digestion or sacrificial materials and carriers which are complex and expensive processes. In addition, current techniques require customized equipment to maintain the delaminated sheets flat as they spontaneously agglomerate into a crumpled 3D rounded shape due to traction forces (Haraguchi, Y., et al., 2012; Tadakuma, K., et al., 2013; Hirose, M., et al., 2000).

### SUMMARY

The present application discloses a technique that allows delamination of cell layers and rapid self-assembly into a cell sheet using a simple pH trigger and without the need for any modified surfaces, enzyme treatments or external stimuli such as electrical and magnetic fields. This technique can be used with cells that are capable of syncytialization and fusion such as skeletal muscle cells and placenta cells. Multiple cell sheets can be attached to each other and form a thicker 3D construct, for example, double- and quadruple-layer constructs as supposed to single-layer constructs. This technique can be used to make thick grafts that can be used for regeneration purposes, as in vitro models, or to produce meat-like tissues as cultivated meat.

Accordingly, provided herein is a method of making a cell construct, comprising:
a) plating a plurality of cells on a substantially flat surface;
b) growing the plurality of cells to at least 80% confluent to form a cell sheet with intercellular linkages;
c) applying a culture medium having a pH of about 5 to about 6.8 to the cell sheet;
d) replacing the culture medium of step c) with a culture medium having a pH of about 7.5 to about 8.5; and
e) replacing the culture medium of step d) with a culture medium having a pH of about 7 to about 7.7,

to obtain a substantially planar untethered cell sheet.

In one embodiment, the plurality of cells are dissociated prior to the plating of the plurality of cells. Optionally, the plurality of cells are dissociated using trypsin or other enzymes, non-enzymatic dissociation agents, or by applying mechanical forces.

In another embodiment, the plurality of cells are differentiated and/or partially differentiated cells.

In another embodiment, the intercellular linkages form by self-assembly.

In another embodiment, replacing the culture medium having a pH of about 5 to about 6.8 comprises transferring the cell sheet to the culture medium having a pH of about 7.5 to about 8.5 and/or replacing the culture medium having a pH of about 7.5 to about 8.5 comprises transferring the cell sheet to the culture medium having a pH of about 7 to about 7.7.

In another embodiment, the method further comprises stacking two or more cell sheets. Optionally, the stacking of the two or more cell sheets is performed prior to the step of replacing the culture medium having a pH of about 7.5 to about 8.5 with a culture medium having a pH of about 7 to about 7.7.

In another embodiment, the method further comprises rolling, folding, and/or crumbling the cell sheet. Optionally, the rolling, folding and/or crumbling is performed prior to the step of replacing the culture medium having a pH of about 7.5 to about 8.5 with a culture medium having a pH of about 7 to about 7.7.

In another embodiment, at least one region of cell non-adhesive material on the substantially flat surface prevents the plurality of cells from attaching to the at least one region.

In one embodiment, the construct retains a defined shape by controlling the location of the initial cell seeding.

In another embodiment, the plurality of cells comprises different cell types existing as a homogenous mixture within the construct.

In another embodiment, the plurality of cells comprises different cell types spatially separated within the construct.

In another embodiment, the method further comprises comprising applying electrical and/or mechanical stimuli to the plurality of cells after plating the plurality of cells.

In another embodiment, the plurality of cells comprise multiple cell types.

In another embodiment, the plurality of cells comprise animal cells, optionally human cells.

In another embodiment, the plurality of cells comprise cells from different animals.

In another embodiment, the plurality of cells comprise hepatocytes, pancreatic Islet cells, fibroblasts, chondrocytes, bone cells, osteoblasts, ondontoblasts, skin cells, keratinocytes, melanocytes, endothelial cells, exocrine cells, smooth or skeletal muscle cells, myocytes, preadipocytes, adipocytes, ectodermal cells, ductile cells, kidney cells, intestinal cells, parathyroid and thyroid cells, nerve cells, ocular cells, retina cells, integumentary cells, immune cells, vascular cells, pluripotent cells and stem cells, cancer cells and tumor cells, or combinations thereof.

In another embodiment, the plurality of cells comprise plurality of cells comprise cells capable of syncytialization.

In another embodiment, the plurality of cells comprise cells capable of syncytialization comprise myoblasts, cytotrophoblasts, choriocarcinoma cells, or combinations thereof.

In another embodiment, the plurality of cells comprise the plurality of cells comprises myoblasts, muscle cells, endothelial cells, fibroblasts, neuroblastoma cells, preadipocytes, adipocytes, or a combination thereof.

In another embodiment, the plurality of cells comprise the plurality of cells comprises myoblasts, preadipocytes, or a combination thereof.

In another embodiment, the plurality of cells comprise the plurality of cells comprise plant cells.

In another embodiment, the method comprises administering the cell construct to a subject in need thereof.

Also provided is a cell construct made according to a method as described herein.

In one embodiment, the cell construct is an artificial meat product.

Also provided is a use of the cell construct for in vivo cell therapy or for research and development, optionally in vitro research and development.

In another embodiment, the construct is for use in vivo for cell therapy or for use in research and development, optionally in vitro research and development or for research and development.

In another embodiment, the construct is used in vivo for cell therapy.

In another embodiment, the construct is used to prepare artificial meat products.

Also provided herein is a cell construct comprising a plurality of differentiated and/or partially differentiated cells formed according to the method described herein.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows a schematic of the A) differentiation, maturation, and sheet formation protocol; B) delamination process: cells are partially differentiated and replated to perform fusion and once cells are fused and show traction on the edges, stepwise treatment with basic (upper right), acidic (upper and lower middle), and neutral media (far left and lower right) is performed to form flat sheets exemplary embodiments of the application.

FIG. 2 shows the A) cell traction force exerted at the edges of the wells; B) advancement of traction to form undefined agglomerated structures that could lead to spheroid-like structures; C) advancement of folding in case sheets are kept in medium A after delamination is completed in exemplary embodiments of the application.

FIG. 3 shows A) single-, double-, and quadruple-layer constructs 1 and 24 hrs after delamination; B) shrinkage pattern of single- and multiple-layer sheets with time—samples are kept in wells of a 48 well plate after delamination from wells of 24 well plates as performed in exemplary embodiments of the application. From left to right: Single, Double, Quadruple.

FIG. 4 shows deformed single-layer construct after 1 day from side and top views (single-layer constructs are more prone to unintended deformation) in an exemplary embodiment of the application.

FIG. 5 shows the difference between shrinkage of sheets formed with 0.235×10⁶ cells in a 24 well plate and 0.480×10⁶ cells in a 12 well plate in an exemplary embodiment of the application. Left: one hour; Right: 24 hours.

FIG. 6 shows A) DAPI and Nile Red stained samples before and after delamination as well as after stacking—staining is performed on monolayer cell culture 1 hr before delamination and on sheets 1 hr after (DAPI stains the nuclei while Nile red stains the cellular lipid); B) fluorescent and brightfield images of quadruple-layer constructs formed with cells stained with DiO and DiI (layers are numbered from bottom to top); C) H&E stained section of a single-layer constructs; D) live/dead stained samples immediately after delamination (top view)—consistent with shrinkage pattern, thickness of sheets increases while cells stay viable during the process—in exemplary embodiments of the application. In A, B and C, from left to right is: Monolayer in GM, Monolayer in DM, Single-layer Constructs, Double-layer Constructs and Quadruple-layer Constructs. In D, from left to right is Without Differentiation and With Differentiation.

FIG. 7 shows difference between compactness of cells A) before and B) after delamination—in accordance with decrease in sheet size, cell compactness increases—in exemplary embodiments of the application. Left is D3 and Right is D4.

FIG. 8 shows assays performed on the day before delamination (D1, monolayer cultures in GM and DM) and the day after delamination (D3, single-, double-, and quadruple-layer constructs) in exemplary embodiments of the application: A) metabolic activity using ABA; B) cytotoxicity of the process using LDH levels in the conditioned medium; C) total protein content of each construct using BCA. Amounts are normalized to GM group and monolayer cultures on plastic surfaces at D1 are shown in patterned fill while cell sheet samples at D3 are depicted in solid fill; D) expression of skeletal muscle cell maturation markers in cells grown in GM and cells undergone differentiation and maturation protocol at the day before delamination (D1). P-value<0.01 for n=4 in an exemplary embodiment of the application; E) immunofluorescence images of control groups (formed in GM and DM) at D3 before delamination and cell sheets after delamination using anti-desmin antibody (samples grown in DM showed higher expression of desmin and formation of thick and multinucleated myofibers that are preserved after delamination) in an exemplary embodiment of the application; F) effect of treatment by cytoskeleton-disruptive drug latrunculin B (100 nM) on partially differentiated cells in DM and undifferentiated cells in GM (treatment is performed at D1 for 1 hr) in an exemplary embodiments of the application.

FIG. 9 shows long-term effect of delamination and stacking multiple layers on cell A) viability and B) cytotoxicity—assays are performed one day after delamination (D3 in the whole process) and two days after (D4 of the process). P-value<0.01 for n=3—in exemplary embodiments of the application.

FIG. 10 shows A) Phase contrast and B) fluorescent stained F-actin (using phalloidin) images of cells without differentiation (in GM), and with differentiation (in DM) taken at D3 before delamination using 10× and 20× magnifications—cells in differentiated state are elongated and has formed thick myofibers—using samples fixed with 2% paraformaldehyde after washing with PBS and staining using 1:40 dilution of phalloidin (Alexa Fluor™ 488 Phalloidin, Thermofisher, A12379) stock solution (300 units in 1 mL methanol) in PBS with 1 hr incubation at room temperature in exemplary embodiments of the application.

FIG. 11 shows delamination of layers after 24 hr of treatment with 50 and 100 nM solution of Latrunculin-B (50 nM treated groups delaminated but were fragile but 100 nM treated ones did not form continuous sheets) in an exemplary embodiment of the application.

FIG. 12 shows A) forming of sheets with annular distribution of cells; B) formation of heterogenous constructs by co-culture of C2C12s and HUVECs with the ratio of 5:1; C) delaminated sheets of BeWo cells (since 100% fusion of cells did not occur, they did not delaminate as a continuous sheet) in exemplary embodiments of the application.

FIG. 13 shows A) forming sheets with blocked regions using Teflon tape, which is highly hydrophobic and allows spaces devoid of medium and cells; B) heterogenous constructs by co-culture of C2C12s and HUVECs with the ratio of 5:1 before delamination and after delamination (compaction of cells after delamination is also evident here) in exemplary embodiments of the application.

FIG. 14 shows A) co-culture of C2C12 cells with NIH-3T3 fibroblasts and SH-SY5Y neuronal-like cells with 5:1 ratio and B) co-culture of C2C12 myoblasts and 3T3-L1 preadipocytes before and after delamination with 1:3 ratio—starting culture with C2C12 having as low as 25% of the initial population is still enough to form continuous sheets with proper integrity—in exemplary embodiments of the application.

FIG. 15 shows a schematic of A) various steps of the proposed process to form cell sheets as building blocks of the lab-grown meat; b) Various protocols used for differentiation and maturation of both myoblasts and preadipocytes to show that different amounts of cells or different treatment protocols can be used to tune protein and lipid content of the samples in exemplary embodiments of the application. Step 1 is in Medium A, Step 2 is in Medium B and Steps 3 and 4 are in Medium N.

FIG. 16 show A) SEM images of 1:0 and 1:3 samples in P3, inset shows the Hematoxylin and Eosin stained cross-section of 1:0 in P3, arrow in 1:0 sample shows the elongated muscle cells while the arrow in 1:3 sample shows the adipocyte that has accumulated lipid droplets; B) brightfield and fluorescent images of samples 1:3 ratio in P3 before and after delamination (C2C12s and 3T3-Lls are stained with different fluorescent dyes); C) protein and lipid content of samples with different initial ratios of partially differentiated myoblasts to preadipocytes undergone different differentiation protocols; D) Comparison of different conditions in terms of protein and lipid contents as well as ease of sheet formation (delamination); E) increase in lipid content of different groups compared to 1:0 samples in P1; F) brightfield images of 1:0 and 1:3 samples in P3 after digestion with collagenase, lipid droplets (arrows) that were preserved during sheet formation process are observable in exemplary embodiments of the application.

FIG. 17 shows sheets formed in A) 24 well plates, B) 6 well plates, and C) 10 cm dishes using 1:0 ratio in P1 were delaminated and stacked to form meat-like structure using this scalable technique to make sheets with different sizes—the more layers are stacked, the less shrinkage happens afterwards; 18-layer stack of sheets formed in 6 well plate showed a low shrinkage after 24 hrs of incubation but a 2 layer stack formed in 24 well plate showed more than 50% shrinkage—in exemplary embodiments of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “construct” or “cell construct” as used herein refers to both a single cell sheet and a plurality of associated cell sheets, for example a multilayer cell sheet.

### II. Methods and Constructs of the Application

The ability to form tissue-like materials that have high cell density with significant amount of cell-cell and cell-ECM interactions is critical in applications such as drug discovery and regenerative medicine. Self-assembly methods can be used to produce tissue like constructs with high cellular density and well-defined tissue architecture in the form of spheroids, organoids, or cell sheets. The cell sheet format is important in regenerative medicine for use as patches that can integrate with surrounding tissues.

Disclosed herein is a pH-induced cell sheet engineering technique that does not need modified surfaces or electrical or mechanical stimuli. After cells are grown to confluence to form cell sheets with intercellular linkages, a stepwise treatment with media that is slightly acidic, basic, and eventually neutral induces delamination as continuous sheets and stops them from agglomeration. These sheets can be stacked to form thicker structures, they can be formed with co-culture of cells that show fusion property with cells that do not show this behavior, and sheets with hollow patterns in them can be formed to make channel-like structures when they are stacked. Different assays were performed to compare viability, function, and maturation of cells in sheets with cells cultured on plastic surfaces in 2D monolayers to show that this technique has minimal effect on cell viability and allows proper function and maturation of cells. Proof of concept with this technique was performed using the C2C12 skeletal cell line as well as the BeWo placenta cell line but it can be used with other cell types with similar behavior. It could be used with 2D cell patterning techniques to prepare sheets with different cell types on the same layer. Compared to other techniques it is inexpensive, easy to perform, and does not need modified surfaces and any other specific equipment as robust individual sheets that can handle significant mechanical forces such as pipetting from one vessel to another or handled with tweezers are produced. This is relevant for scalability using automated robotic handling. Constructs can be used for applications such as tissue engineering and regenerative medicine and as in vitro models for applications such as drug discovery.

Using this method can avoid the use of exogenous scaffolding material or ECM that are required in other lab-grown meat technologies as the cultured cells themselves produce complex ECM that is preserved and makes robust sheet structures with texture. The growth and construction of these as individual sheets and their subsequent parallel assembly into thicker structures avoids mass transfer induced growth limitations and the need for integrated vasculature. Finally, the complex ECM produced in these sheets during growth include biomolecules such as E-cadherin and Laminin 5, which then lend adhesive properties to the individual sheets and enable them to be stacked to form continuous thick constructs.

Accordingly, provided herein is a method of making a cell construct, comprising:
a) plating a plurality of cells on a substantially flat surface;
b) growing the plurality of cells to at least 80%, 85%, 90%, 95% or 100% confluence to form a cell sheet with intercellular linkages;
c) applying a culture medium having a pH of about 5 to about 6.8 to the cell sheet;
d) replacing the culture medium of step c) with a culture medium having a pH of about 7.5 to about 8.5; and
e) replacing the culture medium of step d) with a culture medium having a pH of about 7 to about 7.7,

to obtain a substantially planar untethered cell sheet.

In one embodiment, the method comprises:
c) applying a culture medium having a pH of 5 to 6.8 to the cell sheet;
d) replacing the culture medium of step c) with a culture medium having a pH of 7.5 to 8.5; and
e) replacing the culture medium of step d) with a culture medium having a pH of 7 to 7.7.

The plurality of cells may be plated on the substantially flat surface by any method known in the art. In some embodiments, plating comprises replating from a cell culture plate. The plurality of cells are optionally plated to a cell density of 0.1-10⁷ cells/mL of cell media. As used herein, the term “substantially flat surface” refers to a surface such as a cell culture plate or other substrate surface suitable for cell attachment, for example a porous or non-porous surface, synthetic or natural surface, including but not limited to polycarbonate, cellulose and polycaprolactone. The surface is optionally polystyrene.

The cells may be cultured on any typical cell culture surface prior to the plurality of cells being plated on the substantially flat surface. For example, cells may be grown on porous particles in a bioreactor or in a bioreactor without a scaffold.

After culturing, the plurality of cells are optionally dissociated before being plated on the substantially flat surface. In some embodiments, for example when the cells grown without a scaffold, the cells do not need to be dissociated prior to plating. Cells may be dissociated by any method known in the art, including, but not limited to dissociation using trypsin or other enzymes such as dispase or collagenase, non-enzymatic dissociation agents Accutase™, or by application of mechanical forces such as scraping.

After the cells are plated on the surface, they may be grown to at least 80%, 85%, 90%, 95% or 100% confluence, optionally 100% confluence. As used herein the term “confluence” refers to the percentage of the surface area of a 2D culture that is covered with cells.

In some embodiments, growth of the plurality of differentiated and/or partially differentiated cells to confluency forms a substantially planar cell sheet with intercellular linkages. Optionally, the cells secrete ECM components. As used herein, the term “intercellular linkages” refers to any means by which one cell joins to another cell. Intercellular linkages may be direct or indirect. Intercellular linkages include, but are not limited to, cell-cell adhesion, cell-cell interactions and cell-cell fusion. Intercellular linkages also include interactions between cells and extracellular matrix (ECM) and between cell membrane proteins. In some embodiments, the intercellular linkages form by self-assembly. The intercellular linkages result in the formation of a cell sheet, a substantially planar, sheet-like cluster of cells.

A stepwise treatment with slightly acidic medium followed by slightly basic medium is used to trigger delamination (detachment) of the cell sheet from the culture surface and preservation of the substantially planar sheet-like profile. In some embodiments, treatment with slightly basic solutions following delamination prevents the cell sheet from forming a non-planar configuration. In some embodiments, treatment with a slightly acidic culture medium comprises applying a culture medium to the cell sheet having a pH of about 5 to about 6.8 In some embodiments, the cell sheet is incubated in the slightly acidic culture medium for about five minutes to about 10 minutes at 37° C. In some embodiments, treatment with a slightly acidic culture medium may be accompanied with gentle agitation, such as shaking the plate and/or pipetting the culture medium to accelerate the delaminating process. In another embodiment, a slight shear force may be applied to the edges of the cell sheet to accelerate the delaminating process.

Following treatment with a slightly acidic culture medium, the slightly acidic culture medium is replaced with a slightly basic culture medium. This step may be performed immediately after delamination is complete. In some embodiments, treatment with a slightly basic culture medium comprises replacing the slightly acidic culture medium with a culture medium having a pH of about 7.5 to about 8.5. In some embodiments, the cell sheet is incubated in the slightly basic culture medium for about three to 60 minutes or about five minutes at 37° C.

Following treatment with a slightly acidic culture medium, the slightly acidic culture medium is replaced with a neutral culture medium. In some embodiments, this step comprises replacing the slightly basic culture medium with a culture medium having a pH of about 7 to about 7.7. In some embodiments, the cell sheet is incubated in the neutral culture medium at 37° C. for as long as is needed to keep them alive.

In some embodiments, replacing the culture medium comprises transferring the cell sheet to another culture plate or well comprising the new culture medium, for example using standard pipettes or tweezers. The cell sheet may immediately unfurl and flatten after transfer. In another embodiment, the previous culture medium is removed, for example via pipette and replaced with the culture medium.

As used herein, the term “cell culture medium” refers to a liquid or semi-solid designed to support the growth of cells. In one embodiment, the cell culture medium is a liquid medium. A cell culture medium that is suitable for the specific cell type(s) of the plurality of cells may be used. In one embodiment, the cell culture medium comprises natural biological substances selected from the group consisting of plasma, serum, lymph, amniotic fluid, pleural fluid, growth factors, hormones, crude protein fractions, recombinant proteins, protein hydrolysates, tissue extracts or combinations thereof. In another embodiment, the cell culture medium comprises a basal medium and supplements selected from the group consisting of plasma, serum, lymph, amniotic fluid, pleural fluid, growth factors, hormones, crude protein fractions, recombinant proteins, protein hydrolysates, tissue extracts or a combination thereof. Examples of cell culture media useful in the present methods include, but are not limited to, Dulbecco's Modified Eagle Medium (DMEM), supplemented for example with 10% V/V fetal bovine serum (FBS) and 1% Penicillin-Streptomycin, EBM-2 medium, and McCoy's medium supplemented for example with 15% V/V fetal bovine serum (FBS) and 1% Penicillin-Streptomycin.

In one embodiment, the slightly acidic, slightly basic and neutral cell culture media may be the same media, but at different pHs or they may be different media.

Following the stepwise treatment of acid, basic and neutral culture mediums, the cell sheet remains as a substantially planar sheet detached from any surface (also referred to as “untethered”).

In some embodiments, the method of further comprises stacking two or more detached cell sheets to form a multilayer construct, optionally a double, triple or quadruple layer construct. The multilayer construct is optionally formed by placing sheets on each other and sliding into position. In some embodiments, stacking cell sheets is performed in the slightly basic culture medium before treatment with the neutral medium. In some embodiments, the cells in the sheet form cohesive linkages with each other after stacking. In some embodiments, the layers of sheets form a mechanically robust construct. Stacking two or more detached cell sheets to form a multilayer construct method may be used to create complex 3D cell culture systems that can mimic natural tissue compositions.

In some embodiments, the cell sheet retains a defined shape by controlling the location of the initial cell seeding. In some embodiments, controlling the location of the initial cell plating comprises blocking cell growth in a defined area of the substantially planar plate surface. For example, at least one region of cell non-adhesive material, for example polydimethylsiloxane, on the substantially flat surface may be used to prevent the plurality of cells from attaching to the at least one region.

In one embodiment, the cells are plated in a pattern that defines at least one void. A plurality of cell sheets may be aligned and stacked to provide a three-dimensional void in the construct. Optionally, the three-dimensional void comprises or is a channel, an inlet or an outlet.

In another embodiment, multiple cell types can be used and plated in pattern.

Electrical and mechanical stimuli can also be provided to the assembling tissue to mimic the highly complex structure of the native tissue and accelerate its growth and maturation. In some embodiments, electrical and/or mechanical stimuli may be applied after plating the plurality of cells on a substantially flat surface as the plurality of cells are growing to confluency and/or after achieving confluency. The electrical stimuli can be in the form of a DC voltage of 10 mV to 10V or in the form of an AC voltage which is sinusoidal, pulsatile, triangular or arbitrary waveform. Mechanical stimuli can be in the form of a constant tensile force or a periodic tensile or bending forces. It can additionally be in the form of a constant or periodic varying shear force on the surface of the tissue construct. In some embodiments, the electrical and/or mechanical stimuli may be used together or separately.

In some embodiments, the plurality of cells comprise animal cells. In some embodiments, the plurality of cells comprises hepatocytes, pancreatic Islet cells, fibroblasts, chondrocytes, bone cells, osteoblasts, ondontoblasts, skin cells, keratinocytes, melanocytes endothelial cells, exocrine cells, smooth or skeletal muscle cells, myocytes, preadipocytes, adipocytes, ectodermal cells, ductile cells, kidney cells, intestinal cells, parathyroid and thyroid cells, nerve cells, ocular cells, retina cells, integumentary cells, immune cells, vascular cells, pluripotent cells and stem cells, cancer cells and tumor cells, or combinations thereof.

In some embodiments, the plurality of cells comprises cells capable of syncytialization. In some embodiments, the cells capable of syncytialization comprise myoblasts, cytotrophoblasts, choriocarcinoma cells, or combinations thereof.

In some embodiments, the plurality of cells comprises myoblasts, muscle cells, endothelial cells, fibroblasts, neuroblastoma cells, preadipocytes, adipocytes, or a combination thereof. In some embodiments, the plurality of cells comprises myoblasts, preadipocytes, or a combination thereof. In some embodiments, primary animal cells including satellite cells (muscle stem cells), mesenchymal, embryonic, or induced pluripotent stem cells can be considered for the muscle progenitor cells, while adipose tissue derived stem cells or mesenchymal stem cells can be considered for the fat progenitor cells. Cell lines for both these cell types in meat as well as other cell types, including endothelial cells comprising blood vessels and cell types forming the connective tissue, can be created through genetic or chemical induction and may a better option to reduce variability and contamination risks associated with primary cell harvest. In some embodiments, the plurality of cells further comprises plant cells.

In some embodiments, the plurality of cells comprises different cell types existing as a homogenous mixture or substantially homogenous mixture, resulting in a cell sheet comprising a homogenous mixture or substantially homogenous mixture. In some embodiments, the plurality of cells comprises different cell types which are deposited such that the different cell types spatially separated within the cell sheet. In some embodiments, different cell types are deposited in a defined pattern to obtain spatial separation within the construct.

In another embodiment, the plurality of cells are mixed with small particles, for example particles with a diameter of less than 100 uM and then optionally replated. The particles are optionally synthetic or plant-derived materials, proteins or polysaccharides. In one embodiment, the particles are soy particles. Cells may attach to them while fusing to each other.

The constructs can be broken down to smaller pieces, for example by multiple pipetting or other similar methods, to turn into a liquid bioink that can be injected or printed. The liquid bioink can be mixed with other single cells, ECM materials, or hydrogels.

The method described herein may be used to make meat-like tissues of various sizes and thicknesses. Accordingly, in one embodiment, the plurality of cells comprises adipocytes, skeletal muscle cells and/or other connective tissue cells normally present in muscle tissue. Mixtures of different cell types allow fine tuning of fat and protein content in a fast and scalable fashion. For example, constructs made from only myoblasts may be equivalent to lean meat with comparable protein and fat content, while incorporating adipocyte cells in different ratios to myoblasts and/or treatment with different growth media can result in a 5% (low fat meat) to 35% (high fat meat) increase in the fat content.

The constructs produced using the methods disclosed herein better resemble natural structure of meat, and thicker pieces of cultivated meat with natural texture can be produced, when compared to current technologies. For example, the constructs produced by the presently described methods do not include an exogenous scaffold but rather only the ECM that the cells have produced. Visual appearance of the tissues formed can be adjusted from its current yellowish pink tinge, which is due to the absence of blood and reduced myoglobin production in the culture condition with high oxygen levels, to a natural pinkish tone by culturing cells in low oxygen conditions or by using plant based heme or by addition of extra iron. The process for manufacture of meat-like tissue disclosed herein is scalable and amenable for automation, which are relevant criteria for economic production.

Also provided herein is a cell construct comprising a plurality of differentiated or partially differentiated cells formed according to the method described herein. In some embodiments, the construct is used in vivo or in vitro for research and development. In some embodiments, the construct is used in vivo for cell therapy. In some embodiments, the construct is used to prepare artificial meat products (also known as cultivated meat).

In some embodiments, the construct is used in vitro for research and development, such as for modeling cellular interactions in understanding disease and drug discovery. In some embodiments, the construct is used in vivo for cell therapy, such as tissue grafts and artificial organs for implantation.

The constructs described herein can be used for drug screening. Accordingly, also provided herein is a method for screening for activity of a compound of interest comprising treating a construct as described herein with a compound of interest and observing the effect of the compound on the plurality of cells. For example, a compound of interest may be screened for its effect on the growth rate of the cells, the viability of the cells and/or protein expression in the cells. In one embodiment, different doses of the compound of interest may be studied. The compound of interest is optionally a drug candidate, including for example, a small molecule or a biologics.

The constructs described herein can also be used as in vivo or in vitro bioreactors where cells producing specific biomaterials for example, a protein (for example, an antibody), peptide, hormone (for example, insulin), nucleic acid or lipid are included in the biocompatible gel. Accordingly, in such an embodiment, the methods described herein further comprise culturing the construct and isolating a biomaterial of interest.

The constructs described herein can be further used in regenerative medicine. Accordingly, in such an embodiment, the methods described herein further comprise administering the construct to a subject in need thereof.

Also provided herein are methods of using the constructs of the disclosure as in vitro experimental models.

### EXAMPLES

The following non-limiting examples are illustrative of the present application:

### Example 1. Cell Sheet Formation and Stacking

### Materials and Methods

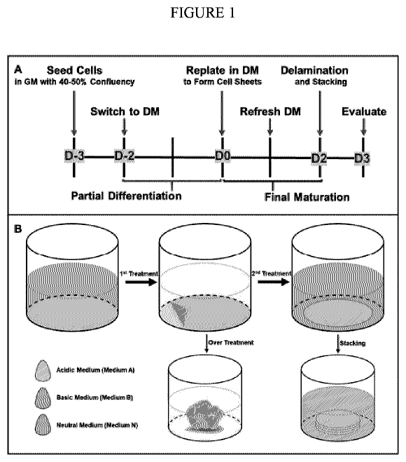

Cell Sheet Formation Process: C2C12 cells were cultured in DMEM (high glucose, with glutamine, Gibco) supplemented with 10% heat inactivated fetal bovine serum (FBS) (US origin, Gibco) and 1% penicillin-streptomycin (10K U/mL, Gibco) in 10 cm dishes until 90% confluent. This medium is the growth medium (GM) for C2C12 cells. At this stage cells were subcultured to new 10 cm dishes in their GM (three days before sheet formation, D-3) to start the new culture with a 40-50% confluency. Next day (D-2) medium was switched to differentiation medium (DM) for C2C12 cells—DMEM supplemented with 2% horse serum (US origin, Gibco), 1% penicillin-streptomycin, and 1% Insulin-Transferrin-Selenium (100×, ThermoFisher). Cells were in this medium for 2 days and then were trypsinized and used to form self-assembled cell sheets (DO). For this purpose, 0.235×10⁶ cells were plated in 24 well plates with 0.5 mL of DM. Medium was refreshed next day (D1) and the day after that delamination process was performed (D2) (FIG. 1A).

In order to start the delamination process DM was replaced with acidic medium (medium A) (DM with 0.1% V/V acetic acid, final pH of ˜6) and samples were incubated for 5-10 min at 37° C. At this point that sheets started to delaminate the process was accelerated by gentle shaking of the well plate. Process could be accelerated by applying shear force using gentle pipetting of medium to the edges of the wells. Immediately after delamination was completed, medium A was replaced with basic medium (medium B, final pH of ˜8) (DM with 0.1% V/V 0.1 M sodium hydroxide in deionized water) in order to prevent samples from forming clumps. After 5 min of treatment with medium B, samples were transferred to neutral medium (medium N, pH of 7.4) (DM without any pH adjustment). In case of stacks, the required number of sheets, either 2 layers of cell sheets (double-layer constructs) or 4 layers (quadruple-layer constructs), were stacked on top of each other by simply dragging them using a tweezer and placing them on each other. This process was also performed in abundant amounts of medium B and after stacking was done, most of the media was aspirated to let the sheets settle down because of their weight for 5 more minutes and then extra medium N was added (FIG. 1B). Images of sheets were taken 1 hr after delamination/stacking and 24 hrs later using a stereomicroscope (Infinity Optical Systems). Diameter of the sheets was used using ImageJ at these time points to study effect of number of layers on shrinkage pattern of the constructs.

Cell Sheet Microstructure: Microstructure of the sheets and their cell distribution was studied by Nile red and DAPI (4′,6-diamidino-2-phenylindole), live/dead, histological staining, and immunofluorescence staining. Before delamination (D2) cells were fixed in 2% paraformaldehyde solution for 1 hr and staining was performed by Nile red (Thermofisher) and DAPI in PBS. One hr after delamination and stacking, single sheets or their stacks were fixed and the same staining was performed. Stock solutions were prepared by dissolving 25 mg Nile red in 1 mL acetone and 10 mg of DAPI in 1 mL PBS. For staining, 10 μL of Nile red and 5 μL of DAPI were dispersed in PBS. After delamination and 1 hr of culture in medium N, live/dead staining was performed on sheets using the kit (ThermoFisher) following the provided protocol. Calcein-AM and ethidium homodimer-1 were diluted in DM and added to the sheets followed by 30 min of incubation. For histological staining samples were fixed, stepwise dehydration and wax embedding was performed followed by sectioning and staining with hematoxylin and eosin (H&E). Immunofluorescence staining was performed for samples grown in GM and DM before delamination at D2 as well as sheets right after delamination for desmin. After fixing, permeabilization was done using 0.2% Triton X-100 in 1% bovine serum albumin (BSA, SigmaAldrich) in PBS for 15 min followed by blocking in 5% BSA for 30 min at room temperature. Treatment with anti-Desmin antibody (Abcam, ab8976) was performed with a 1:500 dilution of the stock solution for 24 hrs at 4° C. followed by 2 hr treatment with 1:1000 dilution of goat anti-mouse secondary antibody (Abcam, Alexa Fluor® 488, ab150113). Images of the samples were taken using an inverted fluorescent microscope (Olympus) with 4, 10, and 20× magnifications in bright field or fluorescent mode and proper filters.

Assessment of Cell Behavior in Sheets: Cellular behavior for undifferentiated and differentiated cells in monolayer format (cells grown in GM and cells undergone the devised protocol) one day before delamination (D1), and cell sheet constructs (single-, double-, and quadruple-layer) one day after delamination (D3) was assessed using Alamar blue assay (ABA) for metabolic activity, CyQUANT™ LDH assay to measure cytotoxicity of the process, Pierce™ BCA Protein assay for total protein content, and a 1-Step qRT-PCR (quantitative reverse transcription polymerase chain reaction) to evaluate differentiation of the cells. All of these kits were purchased from Thermofisher.

Cytotoxicity of the process was evaluated by measuring the lactate dehydrogenase (LDH) levels in the conditioned medium of the sheets. Fresh medium (500 μL) was added to samples the day before LDH assay was performed. Working solution of the assay was prepared according to the instructions on the day of experiment and 50 μL of it was added to a 50 μL sample of the conditioned medium. After 30 min incubation at room temperature, 50 μL of stop solution was added and reading was done immediately using a plate-reader (Tecan Infinite M200 Pro) in absorbance mode at wavelength of 490 nm with absorbance at 680 nm as reference. Five samples in duplicates were used. For ABA, samples' medium was replaced with 400 μL of proper medium (GM for undifferentiated cells and DM for differentiated cells and sheets) containing 10% Alamar blue solution followed by 90 min incubation in dark at 37° C. Two 100 μL aliquots for each sample were used with the plate-reader at excitation/emission of 560/590 nm. Alamar blue containing media of each case were used as control (n=4). Samples were then lysed in 500 μL of 0.5% Triton X-100 in PBS and BCA was performed according to the instructions in duplicates. Lysis solution (0.5% Triton X-100) was used as control. In case of LDH and BCA calibration curve of the kit was prepared to make sure the readings from samples lied in the linear range of the kit. For all of these assays data were normalized to that of cells grown in GM. ABA and LDH assays were performed on single- and quadruple-layer samples 2 days after delamination (D4) as well to study long-term effects of delamination and stacking on cell viability and cytotoxicity.

qRT-PCR was performed on C2C12 cells in differentiation process the day before delamination (D1) and were compared to cells plated at the same day as these cells but in GM (control group). Myogen, MyoD, and (3-Actin were chosen as markers and GAPDH was used as house-keeping gene (Table 1). Samples were lysed using lysis solution of the kit and one-step qRT-PCR kit (Cells-to-CT™ 1-Step Power SYBR™ Green, ThermoFisher) was used following the instructions. The ΔΔCt values for each primer set were calibrated to Ct values of the housekeeping gene and then to the Ct values of control group (n=4 with duplicate readings).

TABLE 1
Sequences of the primers used
for qRT-PCR (5′ to 3′)

| Target | | |
|---|---|---|
| Gene | Forward | Reverse |
| Myogen | GCA ATG CAC | ACG ATG GAC |
| | TGG AGT TCG | GTA AGG GAG |
| | (SEQ ID NO: 1) | TG |
| | | (SEQ ID NO: 2) |
| MyoD | GCC TGA GCA | GCA GAC CTT |
| | AAG TGA ATG | CGA TGT AGC |
| | AG | G |
| | (SEQ ID NO: 3) | (SEQ ID NO: 4) |
| β-Actin | CAT GGA GTC | ATC TCC TTC |
| | CTG GCA TCC | TGC ATC CTG |
| | ACG AAA CT | TCG GCA |
| | (SEQ ID NO: 5) | TA |
| | | (SEQ ID NO: 6) |
| GAPDH | ATG TTT GTG | ATG CCA AAG |
| | ATG GGT GTG | TTG TCA TGG |
| | AA | AT |
| | (SEQ ID NO: 7) | (SEQ ID NO: 8) |

Difference in cytoskeleton reorganization of differentiated cells vs. undifferentiated ones was studied by treating cells with Latrunculin-B (Lat-B) (Abcam, ab144291). C2C12 cells grown in GM and the partially differentiated ones in DM were treated with 100 nM Lat-B in GM and DM, respectively, for 1 hr at D1 and microscopy was performed using inverted microscope in brightfield mode. Effect of cytoskeleton reorganization on detachment of the sheets was also studied by treating the cells with 50 and 100 nM of Lat-B in DM during the 24 hrs before delamination. Lat-B stock solution was prepared by dissolving 100 μg in 1 mL DMSO.

Sheets with Multiple Cells in Co-culture: Heterogenous sheets were formed using co-culture of partially differentiated C2C12 cells and cells from different cell lines including red fluorescent protein tagged human umbilical vein endothelial (HUVEC), green fluorescent protein tagged NIH/3T3 fibroblasts, SH-SY5Y neuroblastoma cells, or 3T3-L1 preadipocytes. These cells were grown in their own media up to 90% confluent (EBM-2 Endothelial Cell Growth Basal Medium for HUVEC, high glucose DMEM supplemented with 10% FBS and 1% pen-strep for NIH/3T3, DMEM/F-12 supplemented with 10% heat-inactivated FBS and 1% pen-strep for SH-SY5Y, and low glucose DMEM supplemented with 10% FBS and 1% pen-strep for 3T3-L1). SH-SY5Ys were stained with DiO fluorescent cell tracker (Thermofisher) for imaging purposes. Partial differentiation of C2C12 was performed the same as before and at the DO, all of the cells were trypsinized and a 5:1 ratio of C2C12 to either HUVEC, NIH/3T3, or SH-SY5Y cells or 1:3 ratio of C2C12s to 3T3-Lls were used to start the coculture. At D2 imaging was done using brightfield and fluorescent modes of the inverted microscope.

Cell Sheets formed with Alternative Fusing Cells: Similar sheet formation and delamination process was performed for placenta choriocarcinoma BeWo cells. Cells were cultured in Ham's F-12K medium containing 10% FBS and 1% pen-strep to 80% confluent. Cells were trypsinized and plated at 0.2×10⁶ cells per well in 24 well plates. One day later medium was switched to the cells' fusion medium (DMEM/F12 containing 10% FBS, 1% penicillin-streptomycin, and 20 μM forskolin (Abcam, ab120058)). Forskolin's stock solution was prepared by dissolving 10 mg in 1.22 mL DMSO. Cells were kept in the fusion medium for 3 more days with refreshing every other day. At day 4, cells were treated with medium A and B the same as before and images of delaminated layers were taken using the stereomicroscope and inverted microscopes.

### Results

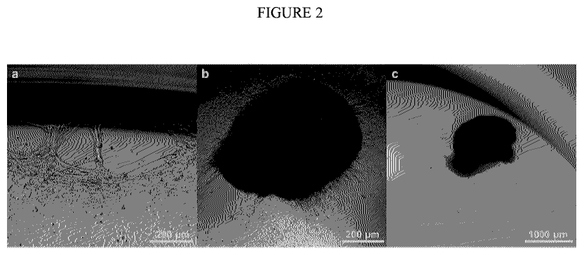

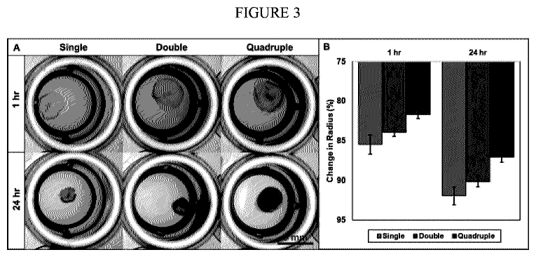

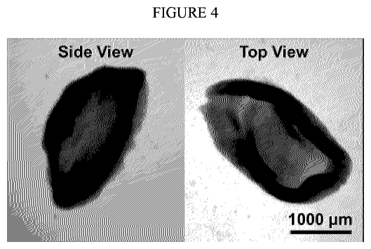

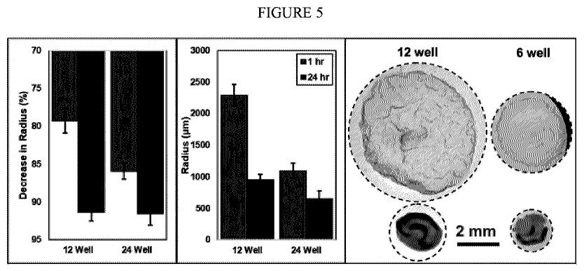

Cell Sheet Formation Process: A technique that uses a simple pH trigger to rapidly detach layers of cells formed on traditional cell culture plates has been developed (FIG. 1A). For this purpose, C2C12 myoblast cells that can differentiate into multinucleated myotubes were used with a defined differentiation protocol. In this protocol, culture initially started in growth medium (GM) at 40-50% confluency (D-3). Medium was changed to differentiation medium (DM) the next day (D-2) and the cells were kept in this medium for two days to prime them towards their final mature and differentiated stage. At this stage (DO) that cells were almost confluent and ready to fuse to each other, they were trypsinized and replated at 0.235×10⁶ cells per well in 24 well plates in DM. In this condition the cells were about 90% confluent and slowly proliferated and fused to each other at the same time. Due to high cell number and metabolic activity of cells, medium was refreshed the next day (D1, one day before delamination) in order to sustain their high metabolic needs and prevent medium to become acidic. At this point the cells fused to form a continuous sheet that applied traction force which was evident due to delamination and curling of the layer formed at the edges of the wells (FIG. 2). This traction could lead to formation of ill-defined agglomerated structures (FIG. 2B) in case no intervention was done to delaminate the sheets in a controlled way. Treating cells with a slightly acidic medium (medium A) at D2 (2 days after replating and 5th day of the whole process) accelerated the delamination process and allowed the cells to rapidly delaminate as a continuous layer. After delamination, if the sheets were kept in the same acidic medium, they would fold upon themselves and formed undefined 3D constructs (FIG. 2C). In order to prevent this, culture media was switched to a slightly basic medium (medium B) which arrested the traction force in the cell sheets and kept them flat. Eventually sheets were transferred to a neutral medium (medium N) as single-layers or double/quadruple-layer constructs (FIG. 1B). Images of these sheets were taken after 1 and 24 hrs and their diameter was measured using ImageJ to study effect of stacking layers on their shrinkage patterns (FIG. 3A). As it is shown in FIG. 3B, single and multiple-layer constructs shrank but the extent of shrinkage was higher when number of layers was lower (single-layer constructs showed 91.94±1.11% shrinkage while quadruple constructs showed 87.06±0.81% shrinkage). Once cells are detached, contractile forces applied by their cytoskeleton was not compensated by the adhesion force from the surface and as a result shrinkage occurs [11, 12]. The lateral shrinkage of the sheet also thickened it and formed a dense 3D-like construct. However, when multiple such layers were stacked on each other immediately upon delamination, the cells formed interlayer attachments that acted as anchorage sites transmitting and compensating the traction forces within each of the layers and as a result, less shrinkage was observed. If this agglomeration was allowed to continue, it could lead to formation of 3D spheroid-like crumpled structures [13]. Interestingly, these crumpled structures had hollow spaces embedded in them which could be used to create 3D cell aggregates with minimal necrotic cores. During this process significant degradation of the sheets was also observed (FIG. 2C). It was also more likely for single-layer constructs to fold on themselves due to the high traction force toward the center of the sheet as they lacked the anchorage sites and were less stable compared to double- or quadruple-layer constructs (FIG. 4). Sheets were also formed in wells of a 12 well plate to show the ability of the technique in making larger sheets using 0.48×10⁶ cells per well and their shrinkage over time was compared to sheets formed in 24 wells (FIG. 5). Suitable pH of media A and B as well as proper timing for the treatment were optimized to be as close as possible to neutral culture and physiological conditions and as short as possible to have the least adverse effect on cell viability. Treating samples with these media for shorter times would not cause the intended effect, while treating them for longer times would lower the cell viability dramatically. For instance, lowering the pH of medium A below 5 and increasing pH of medium B above 8 would result in cell death and degradation of the sheets respectively.

It has been shown that serum depravation by replacing high amounts of FBS with low levels of horse serum can be used for differentiation of C2C12 myoblasts [14]. High levels of insulin (hyperinsulinemia) could help with this process as well [15, 16] and by using such protocols, C2C12 cells have been induced to form the first myotubes after 48-72 hrs [17]. The differentiation medium in this study was devised accordingly. Grabowska et al. reported that in their study C2C12 differentiation in 2D had three different stages, proliferation at day 2, fusion at day 4, and formation of multinucleated myotubes at day 7 [18]. Herein, cells that are ready for fusion are trypsinized (partially differentiated cells that were trypsinized right before fusion) and replated at an almost confluent density. This way they were primed towards their fusing phenotype, but they also showed some degree of proliferation that helped with distribution of the cells to have slightly more cells in the center of the well. This distribution caused the traction force to be radial and resulted in the initial traction and delamination from the edges (FIG. 2A). This technique can be further improved by aligning cells in the wells before performing the delamination. Lam et al. has shown that prealignment of the cells is necessary to get a better fiber formation during myogenesis and to mimic physiological conditions (Lam, M. T., et al., 2009). This technique is valuable in different fields such as skeletal muscle tissue engineering and regenerative medicine where different applications require implantable muscle grafts, or where muscle constructs could be used for biomechanical actuators, lab-grown meat, and drug development (Ostrovidov, S., et al., 2014; Qazi, T. H., et al., 2015).

An interesting aspect of this method is that it does not require any specialized surfaces. Alternate cell sheet engineering methods require either temperature responsive surfaces (Yamada, N., et al., 1990), or dissolving polyelectrolyte sacrificial layers (Guillaume-Gentil, O., et al., 2011) to delaminate which can make the sheets fragile. Similarly, enzymatic methods to sever the attachment of the cells to the surface can also sever the cell-cell linkages and lead to fragile films. Finally, many of the alternate methods require stimuli such as light, electrochemical polarization, ionic solution, or magnetic forces (Owaki, T., et al., 2014) to cause delamination and require specialized equipment. Furthermore, the cell sheets delaminated have to be handled by custom built tools to maintain their flatness as otherwise they would crumple and agglomerate. The method disclosed herein on the other hand, is simple and requires only a quick change in media. Furthermore, it can arrest the traction force on demand and hence maintain the flatness of the sheets. The sheets formed in this way are also mechanically robust and can be easily handled with pipettes or tweezers as opposed to sheets formed using other methods.

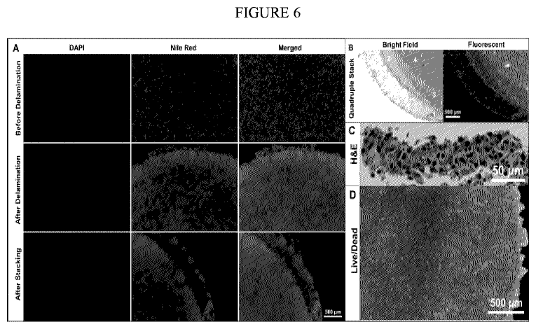

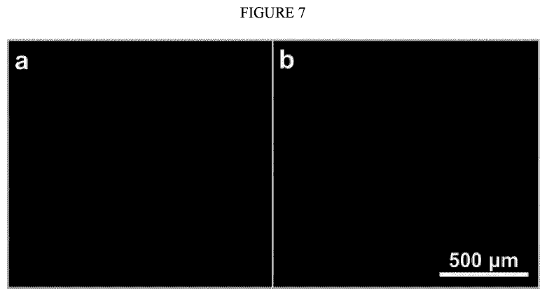

Cell Sheet Microstructure: Differentiation and delamination protocols that have been developed can have an impact on viability of the cells and microstructure of the sheets. For this purpose, different types of staining were performed and imaged using brightfield and fluorescent microscopy. First, Nile red and DAPI staining was performed on cells both before they were formed into cell sheets (D2) and after were delaminated and sheets were formed (FIG. 6A). Consistent with the observed shrinkage of the sheets, cells are more densely packed after delamination (FIG. 7). It is also noticeable that at D1 the whole surface is not covered with cells (confirmed by void spaces in FIG. 6A) while in the sheets there is a continuous and uniform distribution of cells. This is because culture started at 90% confluent at DO and although they were exposed to serum starvation, they slowly proliferated and formed a completely confluent layer by D2 when delamination was performed. Sheets in stacked layers showed robust adherence to each other such that handling them by suction into wide orifice pipette tips with large openings (to avoid damage caused by shear force to the constructs), transfer to another plate, and resuspension in the medium did not cause them to fall apart and maintained their integrity attachment to each other. Brightfield and fluorescent images of quadruple-layer constructs are shown in FIG. 6B demonstrate the scalability of this approach to obtain multilayered sheet like constructs. Even though they are attached to each other the cells from the different layers did not fuse with each other and the attachment can be attributed to cell-cell, cell-ECM, or ECM-ECM adherence between different sheets. Images of cross-section of H&E stained single-layer constructs after 1 day (FIG. 6C) show that each sheet consists of densely packed cells stacked on top of each other with thickness of 40-60 μm. Live/dead staining of sheets 1 hr after delamination (FIG. 6D) shows very few dead cells present indicating that the delamination process did not adversely affect cell viability.

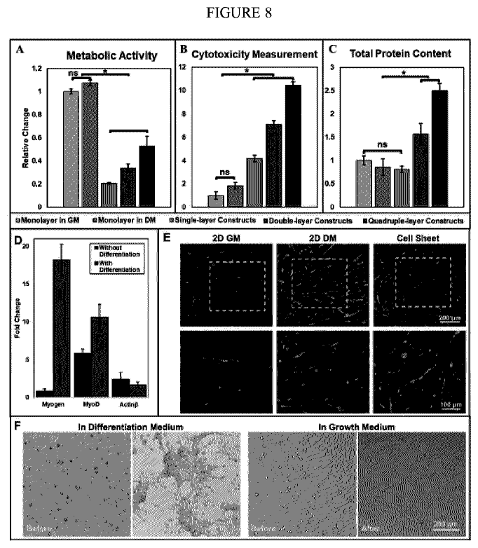

Assessment of Cell Behavior in Sheets: The cell sheet formation and delamination process could potentially have an effect on the cells' function and their viability. In order to understand this effect in greater detail, assays for metabolic activity, total protein content, as well as the cytotoxicity were performed on the cell sheets. These results were compared with similar assays performed on conventional tissue culture plate based monolayer culture of the same number of cells in GM without any delamination (FIG. 8A-C) as a first control group. These control samples were prepared by culturing cells in GM and replating at 0.235×10⁶ cell/well into 24 well plates in GM (the same process that was performed for partial differentiation of cells except the cells were replated in GM instead of DM). The assays were performed one day after culture started (equivalent to D1 of cells that were partially differentiated). Assays were also performed on cells that underwent differentiation process one day before delamination (D1) as a second control group. Metabolic activity of cells in the two control groups at D1 was similar to each other but a dramatic decrease was observed (˜80% decrease) in the case of single-layer sheets. Although total metabolic activity of double- and quadruple-layer constructs was significantly higher than single sheets, this increase was not proportional to the number of sheets, ˜1.65 and ˜2.6 times single-layer, respectively (FIG. 8A). The reduction in metabolic activity of the sheet as compared with the monolayer controls can be due to the change in the form factor of these layers. The surface to volume ratio of the monolayer control is large and the cells in such 2D cultures have sufficient access to nutrients. When the cells are delaminated, they shrink by ˜80-90% and increase in thickness in order to conserve volume. Therefore, their surface to volume ratio dramatically decreases while the cell density dramatically increases. Both of these factors result in transport limitations of nutrients and oxygen that can affect metabolism in the cells. In thicker multilayer constructs one can envision presence of necrotic regions if the cell sheets are tightly adhered to each other which can further reduce their overall metabolic activity per unit volume as manifested in the less than proportional increase of activity in double and quadruple layer constructs.

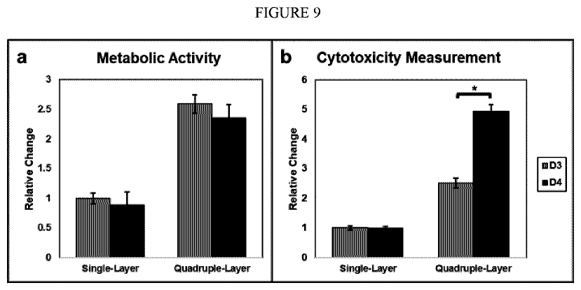

Cells in the second control group (cells grown in monolayers in DM) had similar and very low levels of LDH in their conditioned medium compared to first control group (cells grown in monolayers in GM) which shows the designed protocol to partially differentiate cells had no adverse effect on cell viability. However, these levels were higher for sheets after delamination. This increase in cytotoxicity could be caused by the delamination process (treatment with media A and B) as well as change in cell morphology, loss of their anchorage points, and damage to cell membrane during delamination. The cytotoxicity increased when multiple sheets were stacked (FIG. 8B). As it is shown in FIG. 6C, each cell sheet consists of multiple cells that are tightly compacted on top of each other and is thicker than a monolayer of cells cultured on plastic surfaces. Total LDH levels of double- and quadruple-layer constructs are ˜1.7 and ˜2.5 times of that of a single-layer constructs which again is not exactly proportional to the number of sheets but comparable to fold decrease in their metabolic activity. Total protein content of control groups and single-layer constructs are very close to each other and it increases for double- and quadruple-layer constructs (˜1.95 and ˜3.12 times of single-layer) (FIG. 8C). These findings indicate that the delamination and sheet formation process substantially increase the cell density approaching an in vivo like concentration that leads to reduction in metabolic activity from 2D plate culture. It also shows that stacking multiple layers might impose transport limitations for nutrients and oxygen which might increase cytotoxicity or create necrotic regions. Nevertheless, such single-layer or few layer cell sheets are viable and could be used to mimic in vivo conditions such as high cell density and cellular crowding much better than 2D plate based culture and are of significant interest in regenerative tissue engineering or for use as tissue-analogue models for studying physiological/pathological conditions for example in drug discovery applications (Sakaguchi, K., et al., 2013). Long-term effect of delamination process as well as effect of stacking multiple layers on cytotoxicity was studied by keeping single- and quadruple-layer constructs in culture for one extra day (FIG. 9). Although metabolic activity of both constructs remained the same (FIG. 9a), LDH levels showed a significant increase for quadruple constructs (FIG. 9b). It shows the formed sheets are capable of maintaining their integrity and cell function but stacking multiple layers and increasing the thickness to above mass transfer limits can create cytotoxic environment for cells.

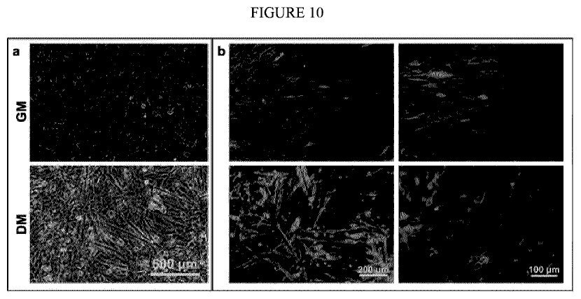

Genetic assays were also conducted to probe for the expression of differentiation/maturation factors. In the process of differentiation of myoblasts, myogenic regulatory markers such as MyoD and Myogenenin are expressed that are indicative of differentiation of the cells from myoblasts into myocytes, myotubes and eventually formation of myofibers (Bentzinger, C. F., 2012). It has been shown that in several cell types including skeletal muscle cells, apoptotic signaling is an important contributing factor (Garrido, C. and G. Kroemer, 2004) and MyoD controls this cell death and apoptosis signaling (Hirai, H., et al., 2010). The expression of Myogenin and MyoD genes was found to be dramatically increased for partially differentiated cells at D1 (the day before delamination) compared to cells in growth medium without any differentiation, as shown in FIG. 8D. (3-Actin, an isoform of actin, is sometimes used as house-keeping gene as it is expressed ubiquitously in most cell types (Perrin, B. J. and J. M. Ervasti, 2010) but its levels can be impacted by the maturation of skeletal muscle cells (Dittmer, A. and J. Dittmer, 2006). During the partial differentiation of the cells however no significant difference was observed between expression levels of partially differentiated cells at D1 and cells in growth medium. Effect of differentiation and delamination protocols on maturation of cells and microstructure of sheets was also studied by staining for desmin, a muscle specific intermediate filament (FIG. 8E). Cells in monoculture with GM showed little expression of this protein while cells in monoculture with DM showed higher expression of the protein as well as formation of thick multinucleated myofibers that are preserved in the cell sheets after delamination. Comparing morphology of cells using bright field images and fluorescent images of samples stained with phalloidin for F-actin (FIG. 10) at D3 before delamination also shows that cells in DM were elongated and formed myotube-like constructs further supporting the finding from genetic assays that the devised protocol is appropriate for differentiation and maturation of these cells.

Effect of differentiation on cytoskeleton reorganization of cells was also studied by treating undifferentiated cells and partially differentiated cells at D1 with Lat-B, an agent that interferes with actin remodeling. Cells were treated with 100 nM Lat-B for 1 hr. Morphology of partially differentiated cells changed very fast to round cells and eventually they formed clumps of cells. Undifferentiated cells however were not affected by this treatment as much and only became slightly round (FIG. 8E). This shows that during the differentiation and fusion process of C2C12 cells, cytoskeleton becomes more active in remodeling and therefore more sensitive to Lat-B.

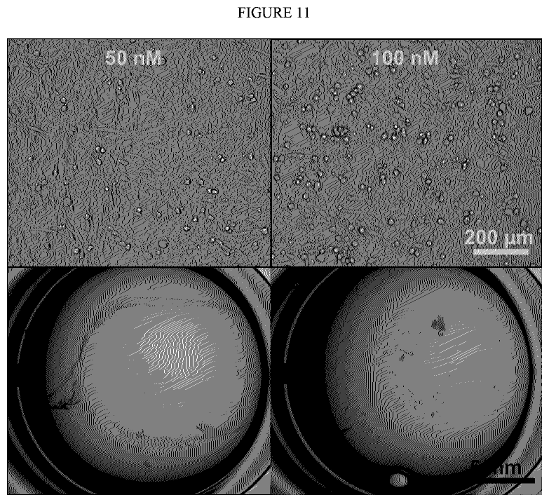

Importance of cytoskeleton reorganization on sheet formation was studied by treating cells 24 hrs before delamination (From D1 to D2) with DM containing either 50 nM or 100 nM. Cells treated with 50 nM Lat-B did not show significant changes in their morphology during the first few hours (data not shown) but after 24 hr of treatment were slightly round (FIG. 11). Cells treated with 100 nM solution after initial dramatic changes, regained their stretched morphology after 24 hrs of treatment but were still rounder compared to untreated cells. Delamination process was performed for both of these conditions, 50 nM treated cells started to delaminate, but the sheets were fragile and broke to smaller pieces. 100 nM treated samples on the other hand did not form sheets at all (FIG. 11). This shows importance of cytoskeleton reorganization on effective delamination of sheets. It is previously shown that treating C2C12 cells with Lat-B causes a decrease in filopodia and lamellipodia extensions during the differentiation process by inhibiting reorganization of cytoskeleton and actin polymerization and therefore reduction in fusion of myotubes (Nowak, S. J., et al., 2009). Nowak et al. reported that C2C12 cells in DM treated with 100 nM had similar morphologies to untreated cells after 1 and 2 days, similar to these observations, but they reported that these cells, although still capable of differentiating, would not fuse to each other (Nowak, S. J., et al., 2009).

The mechanism by which the cell sheets delaminate by acidic medium treatment might be related to loose attachment of differentiated cells to the surface and effect of lower pH on the conformation of ECM proteins. Partial delamination during differentiation has been observed before for other skeletal muscle cell lines such as L6 cells but was considered undesirable for further studies and attributed to incompatible differentiation medium (Lawson, M. A. and P. P. Purslow, 2000). However, herein it has been shown that the cells differentiated properly in the current protocol and fused with each other to form multinucleated fibers that are preserved in continuous layers after delamination. It has been previously shown that during differentiation process of skeletal muscle cells, muscle-specific myosin II will be expressed and replace the non-muscle myosin II that already exists in these cells. These will assemble with F-actin into sarcomers and form branched myotube networks with uncontrolled adhesion that could result in their uncontrolled detachment (Griffin, M. A., et al., 2004) On the other hand, cells adhere to the matrix through focal adhesions mediated by transmembrane integrins which are in turn connected to the internal cytoskeleton (Khalili, A. A. and M. R. Ahmad, 2015). Various studies have shown that by decreasing the pH, cell adhesion to matrix strengthens (Stock, C., et al., 2005). It has been suggested that this could be because of the fact that extracellular acidic conditions promote integrin activation by causing the opening of its headpieces creating a stronger adhesion and attachment to the ECM (Paradise, R. K., et al., 2011). At the same time, it has been shown that in lower pH, cells can show more spread and elongated morphologies (Paradise, R. K., et al., 2011) and form more stress fibers (Faff, L. and C. Nolte, 2000). This elongation in differentiated C2C12 cells that are fused to each other could result in an internal stress in the cell sheet that combined with loosened attachment can result in delamination from the underlying substrate. This is partially supported by the delamination initiating at the edges where the accumulated extensionary forces are the strongest.

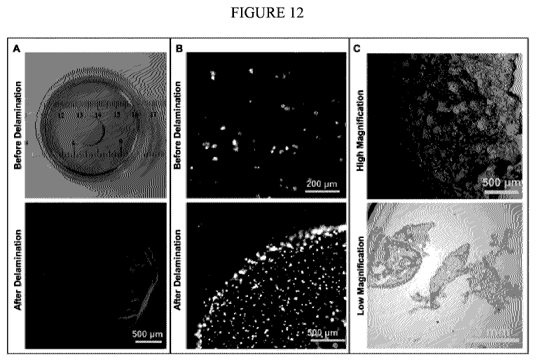

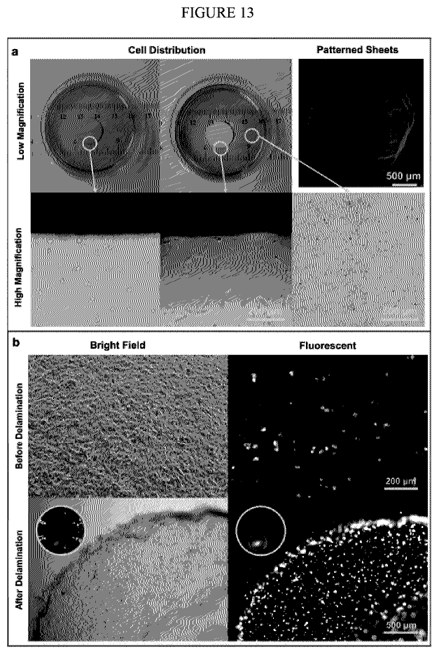

Versatility of Technique on Patterning, Multiple Cells in Co-culture, and Use of Alternative Fusing Cells: The cell sheets can be patterned and shaped by controlling the location of the initial cell seeding. To illustrate this point, a small circular (15 mm diameter) teflon mask was affixed to the bottom of the tissue culture plate (60 cm dish) which prevented the attachment of cells in those regions. A total of 2×10⁶ partially differentiated C2C12 cells in 4 mL of DM were added to plate and next delamination was performed following the same protocol. Continuous sheets were formed, and delamination happened as expected. The delaminated sheets had an annular ring shape instead of the circular shape due to this patterning (FIG. 12A). Such patterned sheets can be stacked on top of each other to form constructs with channels embedded in their structure that can allow perfusion of media through the dense constructs and maintain the viability of cells within. Details of the formation process of such patterned sheets are included in FIG. 13A. Briefly, sheets are formed having regions blocked by using Teflon tape, which is highly hydrophobic and allows spaces devoid of medium and cells. The cells assemble and form sheets only in the region devoid of the hydrophobic tape as the bioink when deposited do not go on top of this region. Even when the entire surface is covered with the cells and media, the cells do not stick on the surface and the pattern can be formed. However, the formation of patterns is more clear and repeatable when the media and cells are restricted from covering the hydrophobic region.

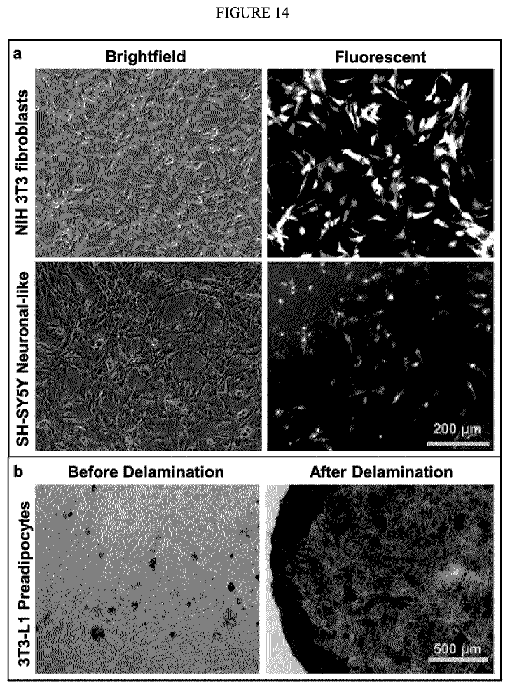

Skeletal muscle cells in vivo have symbiotic interactions with other cell and tissues including vascular tissue, connective tissue, and nervous tissue that may have significance in disease models and in modulating their function (Ostrovidov, S., et al., 2014), for example fibroblasts are important in providing the stability for the skeletal muscle tissue through secretion of ECM components (Chapman, M. A., 2016). Presence of endothelial cells and neuronal cells is also important for vasculogenesis and formation of neuromuscular junctions, respectively. To show the suitability of this technique to recapitulate such interactions, cell sheets of C2C12 and HUVEC were cocultured and delamination was performed the same as before (FIG. 12B). The co-cultured sheets also delaminated cleanly using this technique and can be formed into robust sheets that can be handled. It has previously been shown that co-cultured endothelial cells in cell sheets will reorganize the constructs and migrate towards vascular bed upon implantation to connect the new capillaries and create perfusion networks (Haraguchi, Y., et al., 2012; Sekine, H., et al., 2013). C2C12 cells co-cultured with NIH/3T3 fibroblasts and SH-SY5Y neuron-like cells are also shown in FIG. 9A. The ability of the technique to form sheets with very low numbers of C2C12 cells was also shown by forming sheets where 75% of initial population in the coculture was consisted of 3T3-L1 preadipocytes. These cells differentiated to adipocytes and accumulated lipid droplets that were preserved during the delamination process (FIG. 14B). Although fusion of C2C12 cells to each other to form multinucleated fibers is pertinent to the proposed technique, the ability of the co-cultured sheets to delaminate shows that the establishment of firm junctions with these fusing cells is sufficient to form robust sheets although absence of fusing cells will prevent delamination.

This delamination method for cell sheets is also applicable to other cells that are capable of fusion. Cytotrophoblast cells fuse with each other and form syncytiotrophoblasts in human placenta. BeWo cells, cells from choriocarcinoma and a placenta cell line, show this syncytialization phenomenon and formation of syncytiotrophoblast when exposed to forskolin (Douglas, G. C. and B. F. King, 1990; Ringler, G. E. and J. F. Strauss, 1990). Unlike C2C12 cells that show complete fusion, BeWo cells although capable, don't show complete fusion which has been confirmed using different techniques such as measuring the decrease in E-cadherins and tagging cells to express different fluorescent proteins with different colors and tracking their fusion using fluorescent microscopy, to measure their fusion index (Douglas, G. C. and B. F. King, 1990; Kudo, Y., et al., 2003; Wang, R., et al., 2014). BeWo cells were cultured in wells of 24 well plates and after 72 hrs of treatment with Forskolin, a fusogenic agent, similar delamination process was performed on them. These cells also form cell sheets (FIG. 12C) and delaminated in a faster time period as compared with C2C12 cell sheets. However, the sheets formed were not homogeneous and had small holes in them. This is due to the inability to form a confluent monolayer with 100% cellular coverage and fusion. Nevertheless, this experiment demonstrated that sheets even with non-continuous cell layers and with patterns embedded in them can be delaminated. These sheets did not show much contraction which could be a result of this incomplete fusion or their lower ability in cytoskeleton reorganization.

### Example 2. Layer by Layer Self-Assembly of Meat-Like Tissues

### Methods

Cell culture: C2C12 and 3T3-L1 cells were culture in their growth medium up to 80% confluent. For partial differentiation, culture started with confluency of 40-60% in each cells growth medium which was then switched to the differentiation medium. Cells were kept in this medium for 2 days before they were trypsinized and used for sheet formation. A total cell number of 0.235×10⁶ was used in 24 well plates, 1.15 ×10⁶ cells in 6 well plates, and 6.25×10⁶ cells in 10 cm dishes. C2C12s were cultured in high glucose DMEM supplemented with 10 v/v % heat inactivated (HI) fetal bovine serum (FBS). In their differentiation medium FBS was replaced with 2% horse serum and 1% insulin. 3T3-L1 cells were cultured in low glucose DMEM with 10 v/v % FBS. Their differentiation medium was high Glucose DMEM with 10 v/v % FBS, 1 mg/mL Insulin, 0.5 mM IBMX, 0.25 μM Dexamethasone, and 2 μM Rosiglitazone while their maintenance medium was high Glucose DMEM with 10 v/v % FBS and 1 mg/mL Insulin. All media had 1% penicillin streptomycin and were bought from Thermofisher.

Imaging: Cells were stained with long-term fluorescent trackers (C2C12 with red DiI and 3T3-L1 with green DiO following the supplier's protocol (Thermofisher, catalogue numbers D282 and D275, respectively)) before being partially differentiated. Fluorescent images were taken using an inverted fluorescent microscope (Olympus, USA) using FITC (475-485/485-536 nm) and TXRED (542-582/582-644 nm) filters. For SEM purposes samples were fixed with 2% formaldehyde for 30 min and were critically point dried (Leica Microsystems, Wetzlar, Germany), cut in half, coated with gold and imaged in cross-section and images using TESCAN VP. SEM at 10 kV. For histological staining, fixed samples were dehydrated step wise in 40, 60, 80% ethanol in water and after paraffin embedding and sectioning, staining was done with Hematoxylin and Eosin and images were taken using inverted microscope with 10× magnification.

Cell sheet formation: Partially differentiated cells were replated at specified number for each well size and maturated using any of the three defined protocols (P1, P2, or P3). Delamination process started by treating cells with acidic medium (differentiation medium containing 0.1% V/V acetic acid, final pH of ˜6). This starts the delamination of sheets which can be accelerated by gentle shaking of the well plate or by applying shear force using gentle pipetting of medium to the edges of the wells. after delamination was completed, acidic medium was replaced with basic medium (DM with 0.1% V/V 0.1 M sodium hydroxide in deionized water, final pH of ˜8) in order to prevent samples from forming clumps. After 5 min of treatment with medium B, samples were transferred to neutral medium (differentiation medium, pH of 7.4).

Protein and lipid measurement: Sheets were digested using 500 μL of 2 mg/mL collagenase/dispase (Sigma-Aldrich, catalogue number 10269638001) in PBS for 2 hrs. Lipid content was measured using two 25 μL aliquots of this digest solution in 96 black well plate by addition of 200 μL 0.2% V/V Nile red dye (10 mg/mL in acetone, Thermofisher, catalogue number N-1142) in PBS, following a 15 min incubation, the fluorescent intensity was measured at excitation/emission of 560/640 nm using a platereader (Infinite® M200, Tecan, Männedorf, Switzerland). In parallel, 100 μL of digest solution was lysed using 100 IA of lysis solution (0.5% Triton X-100 in PBS). After 15 min incubation with lysis solution, two 25 μL aliquots were transferred to new 96 well plates and 200 μL of Pierce™ BCA Protein assay (Thermofisher, catalogue number 23227) kit solution (50:1 ratio mixture of solutions A and B of the kit) was added to each well. Absorbance was measured at 562 nm after 30 min incubation. A total of 5 samples were used for each assay (n=5).

### Results

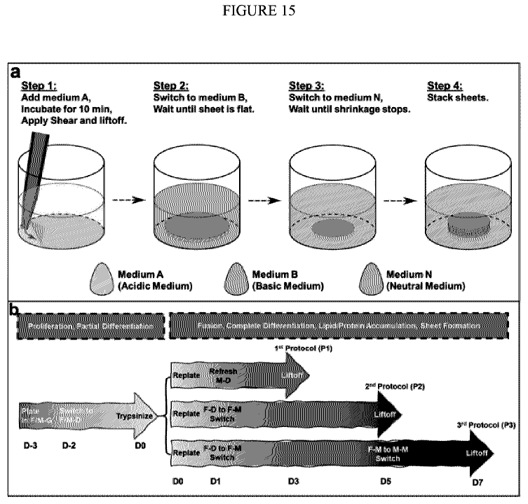

Cell sheet formation and assembly: An approach to rapidly assemble thick slabs of meat tissue from densely packed cell sheets composed of differentiated adipocyte and muscle cells is disclosed herein as shown in FIG. 15A. The process starts with the formation of individual cell sheets. In order to produce adipocyte and muscle co-culture, the progenitor cells (myoblasts and preadipocytes) are grown separately and partially differentiated in their specific differentiation media. Then they are typsinized (before the myoblasts start to fuse) and the appropriate ratio of the partially differentiated adipocyte and muscle cells (corresponding to the desired lipid content in the meat) are plated onto another tissue culture plate at high confluency. The trypsinization and replating does not affect the subsequent full differentiation of them into mature adipocytes and skeletal muscle cells. Upon reaching full confluency, the skeletal muscle cells fuse with each other forming a contiguous sheet which exerts a traction force. They also produce their own ECM that provides additional structure and texture to the sheet. Exposure of this cell sheet to a slightly acidic medium (medium A) leads to its rapid delamination from the culture plate and contraction. The sheet is then exposed to slightly basic medium (medium B) to preserve its flat profile and then transferred into neutral medium (medium N) (FIG. 15A). Finally, several individual sheets are assembled on top of one another whereupon they adhere forming a thick meat-like tissue. This layer by layer process allows formation of highly dense, multicellular, textured tissues that are difficult to produce in other ways. Since the final step can be composed of a large number of sheets assembled in parallel, this process is scalable for fabrication of tissues of any thickness.

Myoblast (C2C12) and preadipocyte (3T3-L1) cell lines were used to demonstrate the feasibility of this approach to assemble meat-like tissue. Different protocols were devised and compared (FIG. 15B) in order to identify conditions that will allow production of different amounts of protein and lipid in the constructs. Different ratios of initial number of cells (C2C12 to 3T3-L1 ratios of 1:0, 1:1, and 1:3) were also used with each of these protocols to eventually produce varying lipid content in the tissues formed. In the 1st protocol (P1) these cells were cultured in muscle differentiation medium (M-DM) for 2 days and delamination was performed at day 3. In the 2nd protocol (P2), cells were treated with fat differentiation and maintenance media (F-DM and F-MM), respectively, for 1 and 4 days before delamination was performed at day 5. In the last protocol (P3) after treating with F-DM and F-MM for a total of 5 days, cells were treated with M-DM for 2 more days and then delamination was performed.

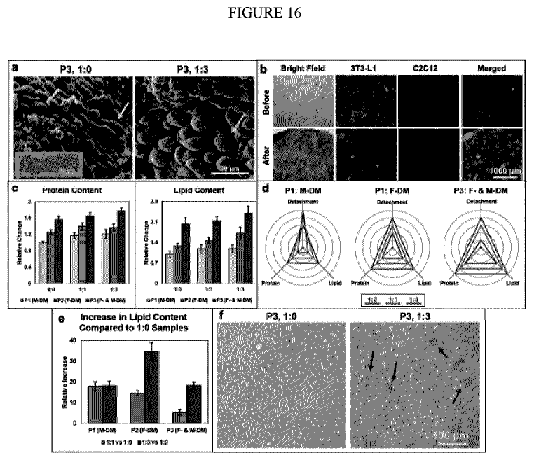

Structural characterization: The microstructure of the cell sheets was studied by scanning electron microscopy (SEM) and light microscopy. SEM images for 1:0 and 1:3 ratios in P3 (FIG. 16A) show the difference between the morphology of cells in each group. In the 1:0 group where only C2C12 cells are present, all of the cells have elongated morphology while in 1:3 group, 3T3-L1 cells that had accumulated lipid are round and bulky. Hematoxylin and Eosin (H&E) staining of the cross-section of the 1:0 group shows the highly dense and compact microstructure of the sheets (FIG. 16A inset) with thicknesses (˜50-100 pin) which is much higher than a single layer of cells in two dimensional culture. Fluorescent staining of the C2C12s with red and 3T3-Lls with green fluorescent dye in a 1:3 ratio sample cultured using P3 shows the presence of both muscle fibers and lipid droplets after delamination which is important to achieve the meat texture and its taste (FIG. 16B). The cellular density and packing also increases significantly after delamination as the sheets shrink and become more compact. Nevertheless, the shrinking process does not damage the fibers or the accumulated fat globules (FIG. 16B). Several cell sheets can be stacked on top of each other simultaneously whereupon they adhere to each other to form thicker structures that better resemble meat structure. Stacked structures resist the traction force induced by the cytoskeleton reorganization which occurs when the sheets are delaminated resulting in lesser shrinkage of the overall structure as compared with individual sheets. Total protein and lipid content of two-layer stacked sheets with various ratios of C2C12 and 3T3-L1 cells were analysed a day after stacking. Samples with highest number of initial 3T3-L1 cells and cultured using P3 protocol had the highest lipid and protein content as shown in FIG. 16C. It was interesting to find that the protein and lipid content of the tissues can be controlled by changing the differentiation protocol as well as the ratio of cells samples. Weight percentage of fat in different types of meat could range from 2-45% depending on the type of meat and its source. In case of beef, lean round can have as low as 6% fat while this amount for lean T-bone is 10% and could go up to 20% for regular beef. In order to compare these samples with actual meat, fresh extra lean beef meat was purchased from local market and samples with similar weight to 1:0 in P1 (˜5 mg) were separated (no fat tissue was observable using naked eye) and the same assays were performed on them. These meat sample had protein and lipid contents that were similar (1.5 and 0.9 times, respectively) to that of 1:0 in P1 sheets. The lower protein content of sheets could be due to cells secreting less ECM once cultured in 2D and proper stimulation could increase the protein content of the sheets. FIG. 16E shows the increase of lipid content in 1:1 and 1:3 groups compared to 1:0 group in each protocol. Using only M-DM (P1) a ˜18% increase was observed for both groups. With F-DM (P2), ˜15 and ˜35% increase was observed for 1:1 and 1:3 groups, respectively. With P3 these increases were ˜5 and ˜20%. Based on these results it can be shown that lipid content of samples is completely tunable compared to samples with only muscle cells in a wide range (5-35%). Note that the lipid measurement is from both cell membrane and lipid droplets in adipocyte cells. The measurement from muscle only cell sheet represents the lipid content of bilipid membrane of the cells while the increases in co-culture with adipocytes can be attributed largely to the stored lipids in adipocytes. From the perspective of manufacturing, the ability to rapidly produce high protein and lipid content as well as the ease of delamination of single sheets to assemble them further, is of importance. Different protocols used in the final growth step and the ratio of cells used in the culture has a significant effect on all of these parameters as shown in FIG. 16D. When M-DM (P1) was used, higher adipocyte number made it more difficult to delaminate the sheets while in the other two protocols, the opposite was observed. The increase in adipocytes lead to an increase in lipid content and the lipid droplets produced were robust and did not break out upon delamination. These lipid droplets don't exist in the muscle only sheets (1:0) while are clearly abundant and are preserved after delamination and digestion of 1:3 groups of P3 (FIG. 16F).

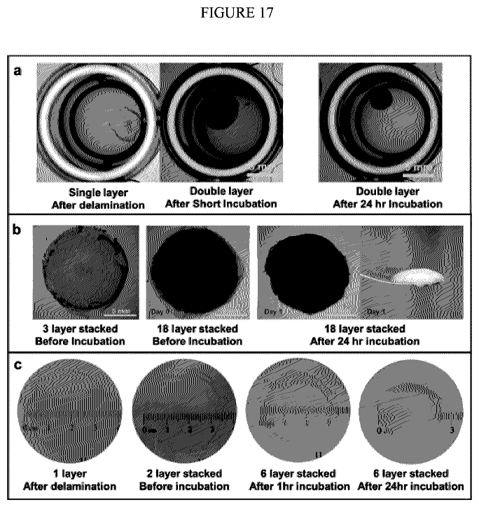

Scale-up process: This manufacturing process is scalable and can be used to make large meat-like tissues in 24 well plates, 6 well plates, or 10 cm dishes (FIG. 13). The ability to form thick and meat-like structures was shown by forming sheets in 6 well plates (1:0 ratio and P1) and stacking 18 layers of them. A multistep assembly process was used wherein three constructs each containing 6 individual cell sheets were formed with 1 hr incubation and later these three constructs were assembled on top of each other and incubated further for 24 hrs (FIG. 17B) (thickness of 1-2 mm can be achieved using this number of layers). Scalability of the technique to form larger structures was further shown by making the cell sheets in 10 cm dishes (1:0 ratio and P1) (FIG. 17C). Individual cell sheets show a significant shrinkage immediately after delamination (from 10 cm to 3.5 cm in case of 10 cm dishes) which helps with creating thicker and stable structures. Once assembled into multilayer constructs the amount of shrinkage is dramatically lower even over longer incubation times (a 6-layer stack shrank from 3.5 cm to 2.5 cm after 24 hrs of incubation) which is important to create stable structures.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Matsuura, K., et al., Cell sheet approach for tissue engineering and regenerative medicine. J Control Release, 2014. 190: p. 228-39.
2. Yamada, N., et al., Thermo-responsive polymeric surfaces; control of attachment and detachment of cultured cells. Die Makromolekulare Chemie, Rapid Communications, 1990. 11(11): p. 571-576.
3. Yamato, M., et al., Thermo-responsive culture dishes allow the intact harvest of multilayered keratinocyte sheets without dispase by reducing temperature. Tissue Eng, 2001. 7(4): p. 473-80.
4. Iwata, T., M. Yamato, and T. Okano, Chapter 29—Intelligent Surfaces for Cell-Sheet Engineering, in Principles of Regenerative Medicine (Second Edition), A. Atala, et al., Editors. 2011, Academic Press: San Diego. p. 517-527.
5. Guillaume-Gentil, O., et al., pH-controlled recovery of placenta-derived mesenchymal stem cell sheets. Biomaterials, 2011. 32(19): p. 4376-84.
6. Owaki, T., et al., Cell sheet engineering for regenerative medicine: current challenges and strategies. Biotechnol J, 2014. 9(7): p. 904-14.
7. Tang, Z., Y. Akiyama, and T. Okano, Temperature-Responsive Polymer Modified Surface for Cell Sheet Engineering. Polymers, 2012. 4(3): p. 1478-1498.
8. Haraguchi, Y., et al., Fabrication of functional three-dimensional tissues by stacking cell sheets in vitro. Nat Protoc, 2012. 7(5): p. 850-8.
9. Tadakuma, K., et al., A device for the rapid transfer/transplantation of living cell sheets with the absence of cell damage. Biomaterials, 2013. 34(36): p. 9018-25.
10. Hirose, M., et al., Creation of designed shape cell sheets that are noninvasively harvested and moved onto another surface. Biomacromolecules, 2000. 1(3): p. 377-81.
11. Shimizu, T., et al., Two-dimensional manipulation of cardiac myocyte sheets utilizing temperature-responsive culture dishes augments the pulsatile amplitude. Tissue Eng, 2001. 7(2): p. 141-51.
12. da Silva, R. M., J. F. Mano, and R. L. Reis, Smart thermoresponsive coatings and surfaces for tissue engineering: switching cell-material boundaries. Trends Biotechnol, 2007. 25(12): p. 577-83.
13. Takezawa, T., Y. Mori, and K. Yoshizato, Cell culture on a thereto-responsive polymer surface. Biotechnology (N Y), 1990. 8(9): p. 854-6.
14. Lawson, M. A. and P. P. Purslow, Differentiation of myoblasts in serum free media: effects of modified media are cell line-specific. Cells Tissues Organs, 2000. 167(2-3): p. 130-7.
15. Conejo, R., et al., Insulin produces myogenesis in C2C12 myoblasts by induction of NF-kappaB and downregulation of AP-1 activities. J Cell Physiol, 2001. 186(1): p. 82-94.
16. Grabiec, K., et al., The influence of high glucose and high insulin on mechanisms controlling cell cycle progression and arrest in mouse C2C12 myoblasts: the comparison with IGF-I effect. J Endocrinol Invest, 2014. 37(3): p. 233-45.
17. Janot, M., et al., Glycogenome expression dynamics during mouse C2C12 myoblast differentiation suggests a sequential reorganization of membrane glycoconjugates. BMC Genomics, 2009. 10: p. 483.
18. Grabowska, I., et al., Comparison of satellite cell-derived myoblasts and C2C12 differentiation in two-and three-dimensional cultures: changes in adhesion protein expression. Cell Biol Int, 2011. 35(2): p. 125-33.
19. Lam, M. T., et al., Microfeature guided skeletal muscle tissue engineering for highly organized 3-dimensional free-standing constructs. Biomaterials, 2009. 30(6): p. 1150-5.
20. Ostrovidov, S., et al., Skeletal muscle tissue engineering: methods to form skeletal myotubes and their applications. Tissue Eng Part B Rev, 2014. 20(5): p. 403-36.
21. Qazi, T. H., et al., Biomaterials based strategies for skeletal muscle tissue engineering: existing technologies and future trends. Biomaterials, 2015. 53: p. 502-21.
22. Sakaguchi, K., et al., In vitro engineering of vascularized tissue surrogates. Sci Rep, 2013. 3: p. 1316.
23. Bentzinger, C. F., Y. X. Wang, and M. A. Rudnicki, Building muscle: molecular regulation of myogenesis. Cold Spring Harb Perspect Biol, 2012. 4(2).
24. Garrido, C. and G. Kroemer, Life's smile, death's grin: vital functions of apoptosis-executing proteins. Curr Opin Cell Biol, 2004. 16(6): p. 639-46.
25. Hirai, H., et al., MyoD regulates apoptosis of myoblasts through microRNA-mediated down-regulation of Pax3. J Cell Biol, 2010. 191(2): p. 347-65.
26. Perrin, B. J. and J. M. Ervasti, The actin gene family: function follows isoform. Cytoskeleton (Hoboken), 2010. 67(10): p. 630-4.
27. Dittmer, A. and J. Dittmer, Beta-actin is not a reliable loading control in Western blot analysis. Electrophoresis, 2006. 27(14): p. 2844-5.
28. Nowak, S. J., et al., Nap1-mediated actin remodeling is essential for mammalian myoblast fusion. J Cell Sci, 2009. 122(Pt 18): p. 3282-93.
29. Griffin, M. A., et al., Adhesion-contractile balance in myocyte differentiation. J Cell Sci, 2004. 117(Pt 24): p. 5855-63.
30. Khalili, A. A. and M. R. Ahmad, A Review of Cell Adhesion Studies for Biomedical and Biological Applications. Int J Mol Sci, 2015. 16(8): p. 18149-84.
31. Stock, C., et al., Migration of human melanoma cells depends on extracellular pH and Na+/H+ exchange. J Physiol, 2005. 567(Pt 1): p. 225-38.
32. Paradise, R. K., D. A. Lauffenburger, and K. J. Van Vliet, Acidic extracellular pH promotes activation of integrin alpha(v)beta(3). PLoS One, 2011. 6(1): p. e15746.
33. Faff, L. and C. Nolte, Extracellular acidification decreases the basal motility of cultured mouse microglia via the rearrangement of the actin cytoskeleton. Brain Res, 2000. 853(1): p. 22-31.
34. Chapman, M. A., R. Meza, and R. L. Lieber, Skeletal muscle fibroblasts in health and disease. Differentiation, 2016. 92(3): p. 108-115.
35. Sekine, H., et al., In vitro fabrication of functional three-dimensional tissues with perfusable blood vessels. Nat Commun, 2013. 4: p. 1399.
36. Douglas, G. C. and B. F. King, Differentiation of human trophoblast cells in vitro as revealed by immunocytochemical staining of desmoplakin and nuclei. J Cell Sci, 1990. 96 (Pt 1): p. 131-41.
37. Ringler, G. E. and J. F. Strauss, 3rd, In vitro systems for the study of human placental endocrine function. Endocr Rev, 1990. 11(1): p. 105-23.
38. Kudo, Y., et al., Quantifying the syncytialisation of human placental trophoblast BeWo cells grown in vitro. Biochim Biophys Acta, 2003. 1640(1): p. 25-31.
39. Wang, R., et al., Live cell imaging of in vitro human trophoblast syncytialization. Biol Reprod, 2014. 90(6): p. 117.

1. A method of making a cell construct, comprising:
a) plating a plurality of cells on a substantially flat surface;
b) growing the plurality of cells to at least 80% confluence to form a cell sheet with intercellular linkages;
c) applying a culture medium having a pH of about 5 to about 6.8 to the cell sheet;
d) replacing the culture medium of step c) with a culture medium having a pH of about 7.5 to about 8.5; and
e) replacing the culture medium of step d) with a culture medium having a pH of about 7 to about 7.7,
to obtain a substantially planar untethered cell sheet.

2. The method of claim 1, wherein the plurality of cells are dissociated prior to the plating of the plurality of cells.

3. The method of claim 2, wherein the plurality of cells are dissociated using trypsin or other enzymes, non-enzymatic dissociation agents, or by applying mechanical forces.

4. The method of claim 1, wherein the plurality of cells are differentiated and/or partially differentiated cells.

5. The method of claim 1, wherein the intercellular linkages form by self-assembly.

6. The method of claim 1, wherein replacing the culture medium of step c) comprises transferring the cell sheet to the culture medium having a pH of about 7.5 to about 8.5 and/or replacing the culture medium of step d) comprises transferring the cell sheet to the culture medium having a pH of about 7 to about 7.7.

7. The method of claim 1, further comprising stacking two or more cell sheets, optionally wherein the stacking two or more cell sheets is performed prior to step e).

8. (canceled)

9. The method of claim 1, further comprising rolling, folding and/or crumbling the cell sheet, optionally wherein the rolling, folding and/or crumbling is performed prior to step e).

10. (canceled)

11. The method of claim 1, wherein at least one region of cell non-adhesive material on the substantially flat surface prevents the plurality of cells from attaching to the at least one region.

12. The method of claim 1, further comprising applying electrical and/or mechanical stimuli to the plurality of cells after plating the plurality of cells.

13. The method of claim 1, wherein the plurality of cells comprise multiple cell types.

14. The method of claim 1, wherein the plurality of cells comprise animal cells, optionally animal cells from different animals.

15. (canceled)

16. The method of claim 1, wherein the plurality of cells comprise hepatocytes, pancreatic Islet cells, fibroblasts, chondrocytes, bone cells, osteoblasts, ondontoblasts, skin cells, keratinocytes, melanocytes, endothelial cells, exocrine cells, smooth or skeletal muscle cells, myocytes, preadipocytes, adipocytes, ectodermal cells, ductile cells, kidney cells, intestinal cells, parathyroid and thyroid cells, nerve cells, ocular cells, retina cells, integumentary cells, immune cells, vascular cells, pluripotent cells and stem cells, cancer cells and tumor cells, or combinations thereof.

17. The method of claim 1, wherein the plurality of cells comprise cells capable of syncytialization, optionally myoblasts, cytotrophoblasts, choriocarcinoma cells, or combinations thereof.

18. (canceled)

19. The method of claim 1, wherein the plurality of cells comprises myoblasts, muscle cells, endothelial cells, fibroblasts, neuroblastoma cells, preadipocytes, adipocytes, or a combination thereof.

20. The method of claim 1, wherein the plurality of cells comprises myoblasts, preadipocytes, or a combination thereof.

21. The method of claim 1, wherein the plurality of cells comprise plant cells.

22. The method of claim 1, further comprising administering the cell construct to a subject in need thereof.

23. A cell construct made according to the method of claim 1.

24. The cell construct of claim 23, wherein the cell construct is an artificial meat product.

25. (canceled)
